(19) United States
(12) Patent Application Publication
(10) Pub. No.: US 2007/0087024 A1
(43) Pub. Date: Apr. 19, 2007

(54) Article comprising fibers embedded in a glassy protein

(76) Inventor: David Philip Knight, Hampshire (GB)

Correspondence Address: INTELLECTUAL PROPERTY / TECHNOLOGY LAW, PO BOX 14329, RESEARCH TRIANGLE PARK, NC, 27709, US

(21) Appl. No.: 10/577,291

(22) PCT Filed: Oct. 29, 2004
(86) PCT No.: PCT/EP04/12295

§ 371 Date: Jul. 14, 2006

(30) Foreign Application Priority Data
Oct. 30, 2003 (GB) 0325354.9

(51) Int. Cl. B32B 27/30; A61F 2/02

(52) U.S. Cl. 424/423; 428/476.6

(57) ABSTRACT

The application relates to an object (30) having a plurality of elongate elements (10) embedded in a matrix (20). The matrix (20) is made from a glassy protein, preferably including serines. The elongate elements can be fibres such as polymers fibrous protein, glass fibres, carbon fibres, carbon nanotubes and montmorilonite clay particles. The application also discloses a method of making the object (30) and a laminate (50) therefrom.

### FIELD OF THE INVENTION

The invention relates to an object comprising a plurality of fibres embedded in a glassy protein.

### PRIOR ART

Sericin is generally considered a waste product in the paste and is often thrown away. There are, however, references in the patent literature to the use of sericin for particular applications.

For example, U.S. Pat. No. 5,728,461 (Nogata et al, assigned to Seriren) teaches the coating of a fibre with sericin. The fibre is dipped, coated or sprayed with an aqueous solution of the sericin. According to this patent, a fibre product is obtained which has improved skin care properties. The fibres described in this patent application are woven into clothing, such as women's tights and are not embedded within a matrix made of sericin.

Similarly Japanese Patent Application Publication No 2000345472 (Hashimoto) also teaches a textile which has been coated with a protein solution containing sericin and/or fibroin. There are again no teachings in this application that the fibres can be embedded in a matrix of sericin.

Knowledge of the use of silk solutions to coat fibres is, however, old technology and dates back to the nineteenth century as is illustrated by German Patent Nr. 7275 issued to Magnier et al. This patent teaches the dissolution of silk in amino acid in order to obtain a solution which is then coated with the solution. The cotton fibres are placed in a bath of the solution and a current applied so that silk is deposited on the surface of the fibre.

Another Japanese Patent Application Publication No. 03284337 teaches a polymer film which is used as a separating membrane. It is made of a thin film of sericin which is cross-linked using a cross-linking agent. The polymer film has no fibres embedded within it.

A wound dressing material made from an amorphous film containing fibroin and sericin as its main component is described in EP-A-0 920 875 (Tsubouchi).The amorphous film is made from regenerated silk solutions. This application does not, however, teach the use of fibres embedded within the film.

As discussed above none of these documents discloses fibres embedded with a matrix made of a protein.

### SUMMARY OF THE INVENTION

The invention comprises an object which has a plurality of elongate elements embedded in a matrix in which the matrix comprising at least a first glassy protein.

The term elongate elements in this context is intended to encompass all types of elongate or longitudinal elements including, but not limited to, filaments, yarns, threads, chopped fibres, short fibres as well as artificial and natural fibres. The term does not imply that the fibres are necessarily longitudinally oriented within the matrix.

The term glassy protein is intended to encompass all proteins which are capable of forming a glass. The dry glass-rubber transition temperature (Tg) of the first glassy protein is greater than 70° C. and preferably greater than 85° C.

The first glassy protein in one embodiment of the invention can be one of the following group of proteins: natural proteins, recombinant proteins and protein analogues. A mixture of these proteins is possible.

The first glassy protein moreover preferably comprises at least 5 mol % of serine or in which at least 5% of the amino acid residues of the first glassy protein are serines. In one embodiment of the invention at least 5% of the amino acid residues of the first glassy protein are phosphorylated. In another embodiment of the invention molecules of the first glassy protein are glycosylated, on average, at fewer than five amino acid residues per molecule.

The amino acid sequence of the first glassy protein comprises at least one domain within which one or more amino acid sequences are repeated at least once. This domain comprises in one embodiment of the invention the 8-residue amino acid sequence:

SEQ ID NO:1

Ser Ser Asn Thr Asp Ser Asn Ser.

In another embodiment of the invention, the domain comprises the 22-residue amino acid sequence:

SEQ ID NO:2

Ser Ser Xxx Ser Xxx Asn Xxx Xxx Val Ser Xxx Thr
Gly Ser Ser Ser Asn Thr Asp Ser Asn Ser.

In yet a further embodiment of the invention, the domain comprises the 37-residue amino acid sequence:

SEQ ID NO:3

Gly Ser Ser Thr Ser Gly Gly Xxx Xxx Ser Ser Thr
Tyr Gly Tyr Ser Ser Asn Ser Arg Asp Gly Ser Val
Ser Ser Thr Gly Ser Ser Ser Asn Thr Asp Ser Asn
Ser.

In a still further embodiment of the invention, the domain comprises the 40-residue amino acid sequence:

SEQ ID NO:4

Gly Ser Ser Thr Ser Gly Gly Xxx Xxx Ser Ser Thr
Tyr Gly Tyr Ser Ser Asn Ser Arg Asp Gly Ser Val
Ser Ser Thr Gly Ser Ser Ser Asn Thr Asp Ser Asn
Ser Asn Ser Xxx

The amino acid sequence can be repeated at least once and preferably several times within the domain. Preferably the domain has a substantially beta-sheet structure.

The first glassy protein can be selected from the group of repetitive block proteins consisting of: sericin, Pseudomonas seringae ice nucleation protein INAZ, Drosophila putative chitin binding protein QVEL9, and from homologues and/or analogues of these proteins. In one embodiment of the invention, the sericin is derived from the cocoon silk of domesticated or wild silkworms.

The matrix in which the fibres are embedded can also comprise a further, second glassy protein which preferably has the same amino acid sequence to the first glassy protein but differs from said first glassy protein by at least 25% in its degree of phosphorylation and/or glycosylation.

The matrix further may also comprise a further polymer material such as, for example, a synthetic or natural rubber.

For certain applications, the inventive object is provided with a coating that is substantially impermeable to water and water-vapour. This protects the matrix material from degradation.

The elongate elements embedded in the matrix can be silk cocoons of domesticated or wild silkworms or layers delaminated from silk cocoons of domesticated or wild silkworms.

Some further examples of the elongate elements which can be embedded within the matrix include, but are not limited to, glass fibres, carbon fibres, carbon nanotubes, and montmorilonite clay particles. The elongate elements could also include a synthetic polymer, a natural polymer or mixtures thereof. Examples of the polymers include but are not limited to polypeptides, polyesters, polyamide, polyarimide, polyvinylchloride, polytetrafluoroethylene, and/or polyurethane polymers. Examples of polypeptides include but are not limited to spider silk proteins, analogues of spider silk proteins, silk-worm proteins, analogues of silk-worm proteins, regenerated silk protein, and mixtures of these proteins as well as fibrous proteins in general. The proteins can be natural or recombinant proteins.

The invention also comprises a method of manufacturing an object with the steps of: preparing, in an aqueous solvent and at a concentration of at least 20 wt %, a solution of a protein capable of forming a glassy state;

contacting a plurality of elongate elements with said solution so that said elongate elements are substantially wetted by said solution;

Substantially filling interstices between said elongate elements with said solution; and rapidly drying the product. This method produces the object with elongate elements embedded in a matrix comprising a glassy protein.

Preferably the solution is obtained by dissolving substantially purified sericin in an aqueous solvent to a concentration of at least 20 wt %.

The invention moreover comprises a method of manufacturing a laminate with the steps of: preparing, in an aqueous solvent and at a concentration of at least 20 wt %, a solution of a protein capable of forming a glassy state;

preparing a plurality of layers of substantially planar sheets, said substantially planar sheets comprising a plurality of elongate elements;

contacting said substantially planar sheets with said solution so that said elongate elements are substantially wetted by said solution;

Substantially filling interstices between said elongate elements and interstices between the sheets of said plurality of substantially planar sheets with said solution; and rapidly drying said substantially planar sheets.

The laminate can be generated as a continuous product in a format selected from the group of formats consisting of a sheet, a ribbon, or a tube.

### DESCRIPTION OF THE DRAWINGS

FIG. 1 shows an example of the invention

FIG. 2 shows an example of the invention with a coating

FIG. 3 shows an example of a laminate made using this invention

FIG. 4 shows an example of the manufacture of the laminate of this invention.

FIG. 5 shows the consensus repeat structure of the sericin precursor protein.

FIG. 6 shows the structure of the ice nucleation protein.

FIG. 7 shows the structure of the consensus repeat structure of the sericin precursor protein.

FIG. 8 shows the results from an LALIGN alignment of the sericin precursor protein with the ice nucleation protein

### DETAILED DESCRIPTION OF THE INVENTION

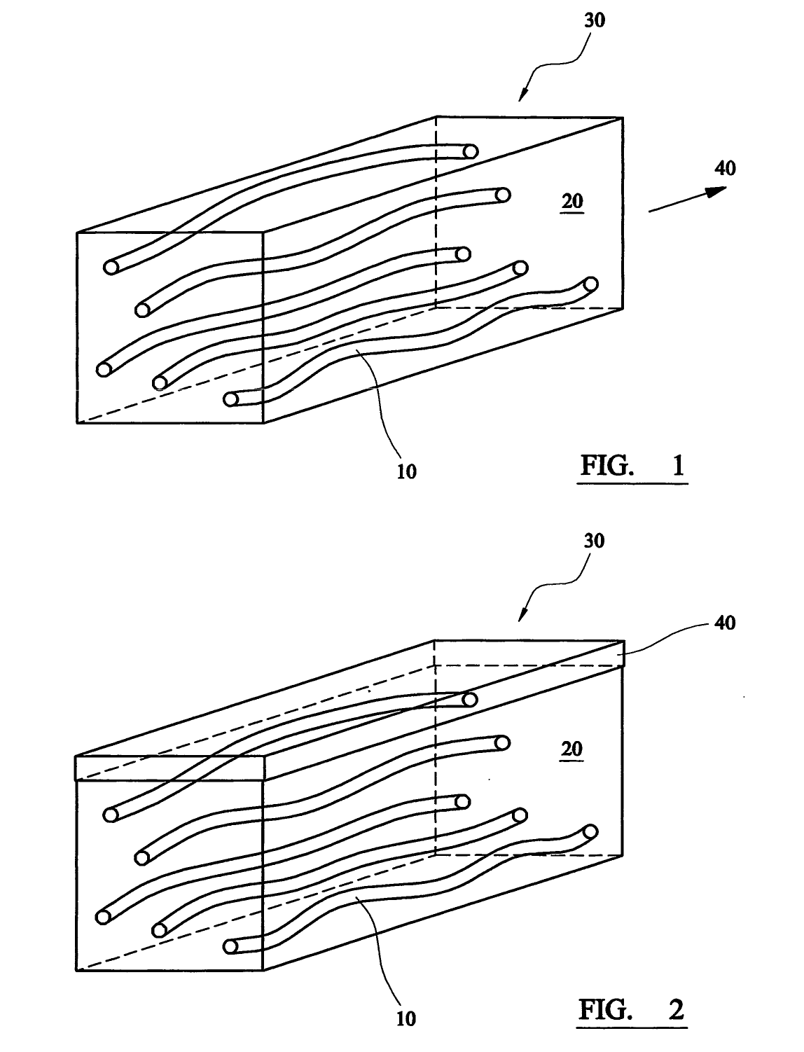

FIG. 1 shows an example of the invention having elongate elements 10 such as fibres embedded within a matrix 20 and forming an object 30. The matrix 20 is made of a protein which might be a natural protein, a recombinant protein or a protein analogue. A mixture of these proteins is possible.

The elongate elements 10 can be—but are not necessarily—oriented substantially along a longitudinal axis 40 of the object 30. For example, the elongate elements 10 can also be wound, combed, embroidered or woven before incorporation into the matrix 20.

Examples of the proteins from which the matrix 20 can be made include but are not limited to sericin, Pseudomonas seringae ice nucleation protein INAZ, Drosophila putative chitin binding protein QVEL9 and from homologues and/or analogues of these proteins. These include proteins containing derivitised and/or synthetic amino acid residues. The sericin protein is derived in one example of the invention from the cocoon silk of domesticated or wild silkworms. As is described in the prior art, this sericin has in the past typically been discarded.

The matrix 20 is preferably made from a protein having at least 5 mol % of serine or in which at least 5% of the amino acid residues of the first glassy protein are serines. In one embodiment of the invention at least 5% of the amino acid residues of the matrix 20 are phosphorylated. In another embodiment of the invention molecules of the first glassy protein are glycosylated, on average, at fewer than five amino acid residues per molecule.

In the preferred embodiment of the invention, the amino acid sequence of the protein from which the matrix 20 is made comprises at least one domain within which one or more amino acid sequences are repeated at least once.

This domain comprises in one embodiment of the invention the 8-residue amino acid sequence:

| | |
|---|---|
| Ser Ser Asn Thr Asp Ser Asn Ser | (SEQ ID NO: 1) |

In another embodiment of the invention, the domain comprises the 22-residue amino acid sequence:

(SEQ ID NO:2)

Ser Ser Xxx Ser Xxx Asn Xxx Xxx Val Ser Xxx Thr
Gly Ser Ser Ser Asn Thr Asp Ser Asn Ser

In yet a further embodiment of the invention, the domain comprises the 37-residue amino acid sequence:

(SEQ ID NO:3)

Gly Ser Ser Thr Ser Gly Gly Xxx Xxx Ser Ser Thr
Tyr Gly Tyr Ser Ser Asn Ser Arg Asp Gly Ser Val
Ser Ser Thr Gly Ser Ser Ser Asn Thr Asp Ser Asn
Ser

In a still further embodiment of the invention, the domain comprises the 40-residue amino acid sequence:

(SEQ ID NO:4)

Gly Ser Ser Thr Ser Gly Gly Xxx Xxx Ser Ser Thr
Tyr Gly Tyr Ser Ser Asn Ser Arg Asp Gly Ser Val
Ser Ser Thr Gly Ser Ser Ser Asn Thr Asp Ser Asn
Ser Asn Ser Xxx.

The amino acid sequence can be repeated at least once and preferably several times within the domain. It is found that the domain has a substantially beta-sheet structure.

In a further embodiment of the invention, the matrix 20 in which the fibres 10 are embedded can also comprise a further protein which preferably has the same amino acid sequence to the first protein but differs from the first protein by at least 25% in its degree of phosphorylation and/or glycosylation.

The matrix 20 further may also comprise a further polymer material such as, for example, a synthetic or natural rubber.

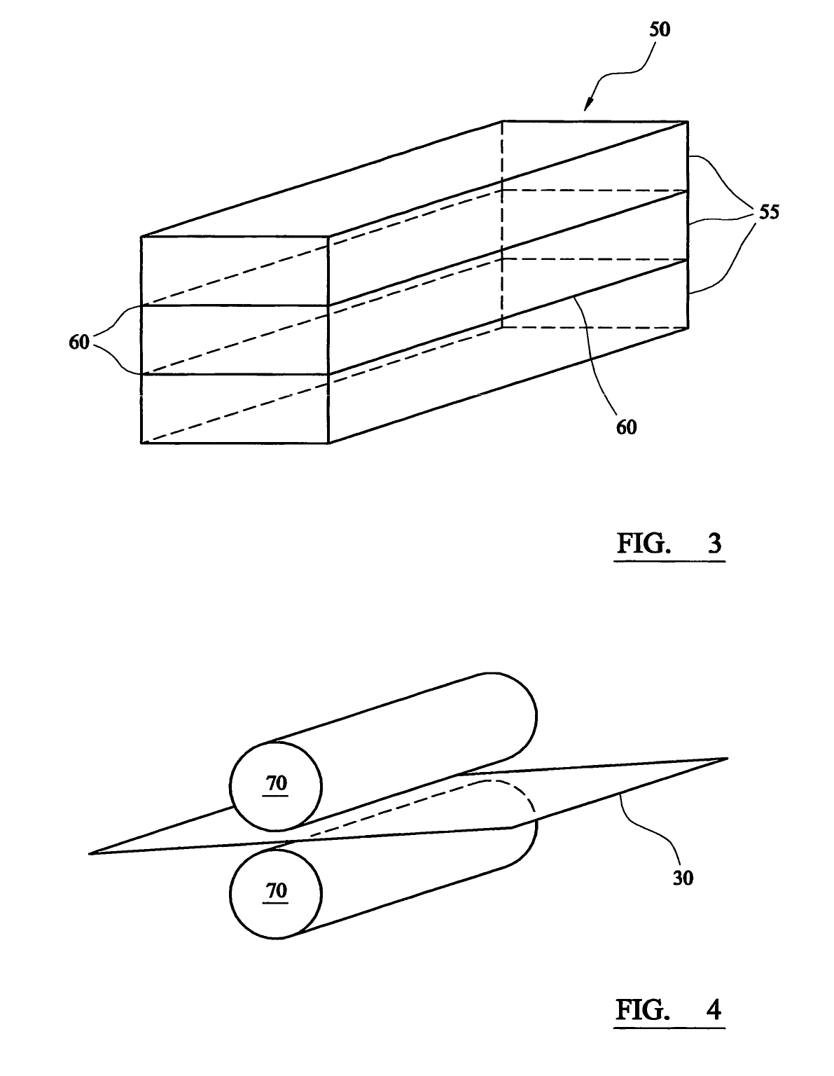

FIG. 2 shows a further embodiment of the invention in which the object 30 is provided with a coating 40 that is substantially impermeable to water and water-vapour. This protects the matrix 20 from degradation. The coating 40 is preferably made from a thermoplastic.

The fibres 10 embedded in the matrix 20 can be made from silk cocoons of domesticated or wild silkworms or layers which are delaminated from silk cocoons of domesticated or wild silkworms.

Other examples of the elongate elements 10 embedded within the matrix 20 include glass fibres, carbon fibres, carbon nanotubes, and montmorilonite clay particles. The elongate elements 10 could also include a synthetic polymer, a natural polymer or mixtures thereof. Examples of the polymers from which the elongate elements 10 are made include but are not limited to polypeptides, polyesters, polyamide, polyarimide, polyvinylchloride, polytetrafluoroethylene, and/or polyurethane polymers. Examples of polypeptides include, but are not limited to, spider silk proteins, analogues of spider silk proteins, silk-worm proteins, analogues of silk-worm proteins, regenerated silk protein, and mixtures of these proteins as well as fibrous proteins in general. The proteins can be natural or recombinant proteins.

A further embodiment of the invention is shown in FIG. 3 which comprises a laminate 50 which is formed by a plurality of sheets 55 in planar form. The laminate 50 can be laminated using the same using the material in the matrix 20, but can also laminated together using an optional adhesive layer 60. The optional adhesive layer 60 can be a synthetic or natural rubber.

The object 30 of the invention is manufactured using the following method. A solution of the protein forming the matrix 20 was prepared in an aqueous solution with a concentration of at least 20% by weight of the protein. The fibres 10 were then placed in contact with the solution so that they are substantially wetted by the solution and the spaces or instices between the fibres 10 are substantially filled by the solution. The product thus formed is rapidly dried so that the object 30 is formed as a composite of the protein which forms the matrix 20 and the fibres 10.

In the preferred embodiment of the invention, the protein forming the matrix 20 is as mentioned above substantially purified sericin.

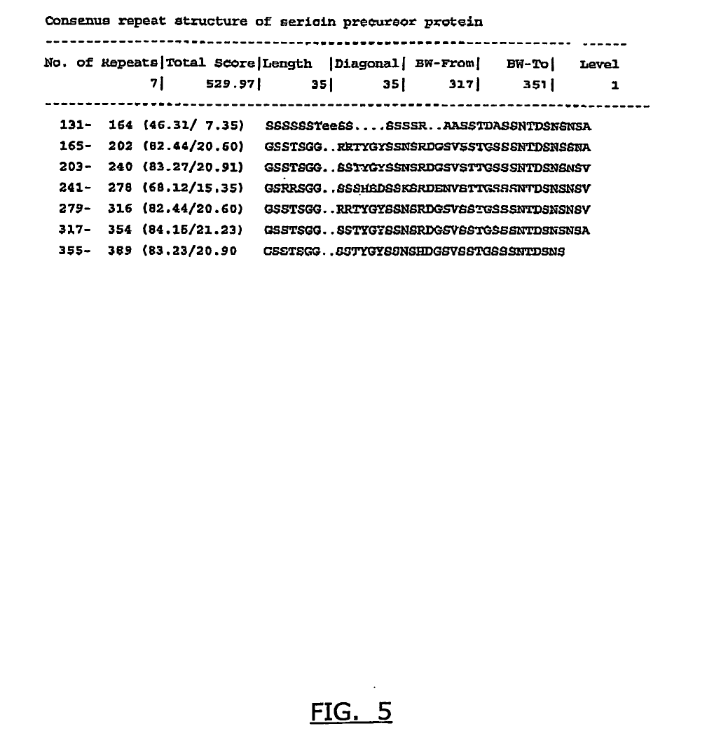

It can be advantageous in making the object 30 to apply pressure to the solution whilst it is drying or after it is substantially solidified. This removes the number of voids that might be formed within the matrix 20 during the drying step. Pressure can be placed, for example, by passing the object 30 through compression rollers 70 as is shown in FIG. 4. The compression rollers 70 can be heated to dry the object 30 quickly. The compression rollers 70 can also be made of a permeable material to allow the passage of water from the object 30.

The drying step can be advantageously performed by vacuum infiltration, by drying using phosphorous pentoxide, by using a flow of dry and/or heated gas.

Prior to foal solidification, the user can place the object 30 into a mould in order to form a desired shape.

Silicate or calcium ions can be added to the solution from which the matrix 20 is formed. These will be incorporated into the matrix 20 and will thereby increase the glass transition temperature of the protein from which the matrix 20 is formed.

The sheets 55 from which the laminate 50 of FIG. 3 are made in an identical manner. The sheets 55 are then coated in one embodiment with the solution from which the matrix 20 is formed or with a further adhesive 20 in order to join the sheets 55 together. As is well known in the art, lamination is improved by applying pressure to the sheets and this can also be done using compression rollers 70 as described above.

The laminates 50 can then be left in a sheet-like form or made into ribbons and tapes, depending on the requirements.

The object 30 and laminate 50 have a variety of uses, for example in the manufacture of a medical product, a wound dressing, a medical implant, a medical prosthesis.

Furthermore, the object 30 and laminate 55 can be used for ballistic protection.

### EXAMPLES

### Example 1

The silk composite material, with additional sericin, forming the object was prepared as follows: The loose silk was stripped from the outside of fresh Bombyx cocoons and the cocoon flattened. Rectangular strips (15×25 mm) were cut parallel to the long axis of the cocoon and infiltrated with a 50% w/v solution of sericin in distilled water. The strips were stuck with outer sides together with a small excess of sericin and dried (1 week at room temperature) between wooden blocks tightly clamped together with a large G-clamp. This material is hereafter referred to as a sericin/native silk composite. Identical strips arranged back to back but not stuck together with sericin were used as a control. Material was then subjected to tensile testing in a Hounsfield mechanical testing instrument. The mean ultimate tensile strength of the sericin composite 25.1 MPa±3.5, n=8 was nearly twice that of the cocoon control 14.9±5.1, n=8 and highly significantly larger (p =0.00021; t-test assuming unequal variances).

### Example 2

To observe final instar silkworms spinning and obtain samples of silk spun on glass, microscope slides were taped together to form glass walled observation chambers with tops and bottoms measuring approximately 75×75 mm and sides 26 or 52 mm high. After observing spinning behaviour under a stereomicroscope the microscope slides were disassembled and the attached silk viewed under polarizing and differential interference microscopes. Samples of silk were also mounted on stubs and sputtered with gold for scanning electron microscopy (SEM).

Silkworms frequently start cocoon construction by crawling forward slowly, moving their heads from side to side. The produces silk fibres laid down on the microscope slide in a pattern formed from numerous elongated figures of eight or from more open sinuous curves whose long axes in both case were approximately at right angles to the direction of travel of the silkworm. An additional piece of behaviour was observed during the formation of the initial loose mat of fibres that surround the cocoon. The silkworm's head sometimes moved away from a glass surface apparently making exploratory movements. During these movements anchoring fibres were formed as follows: The silkworm extended its head out to a surface some distance from its current place of attachment to a matt of silk. This drew out a remarkably long anchoring filament running from the initial mat to the newly contacted surface. Having drawn out the anchoring filament, the silkworm turned its head frequently moving it only short distances from the initial point of attachment of the anchoring filament.

During this behaviour the spinneret remained in contact with the new surface to form an anchoring plaque some 2-3 mm in diameter. This was followed by a rapid drawing of the head away from the attachment plaque often following the course of the first anchoring filament. Repetition of this behaviour produced a strong anchoring thread composed of up to 6 baves attached to a small but well fastened attachment plaque. Moderate numbers of these structures served to anchor the cocoon very firmly to the surrounding surfaces. The toughness of this attachment is thought to depend in part on the multi-stranded, strong anchoring threads and the large areas of sericin stuck to the substratum in the attachment plaques (see below).

Occasionally silkworms failed to spin a normal cocoon but produced instead a considerably larger thin sheet of silk. These provided useful preparations for studying the structure of the cocoon. The scanning electron microscope showed that this sheet of silk consisted of a meshwork of baves often adhering where they crossed or ran close to each other. The appearance of these adhesions suggested that they were formed by a fluid coating of sericin sticking the baves together. A similar appearance was seen where fibres adhered to one another in the cocoon wall. In the tight turns of the figure of eight pattern or attachment plaques a broad, thin area of adhesion of bave to the glass suggested that the liquid sericin had strongly wet glass and spread some distance before it dried.

These observations demonstrate that the wall of the cocoon has a composite network construction consisting of double brins of fibroin cemented together by sericin.

### Example 3

Thin sheets of cocoon wall silk were obtained as described in Example 2.

The thin sheets of cocoon wall silk were slowly strained to fracture between two pairs of pliers. The fractured edges of the thin sheet were mounted on an SEM stub. Controls of thin sheets which were not subject to mechanical strain were also mounted on stubs. After sputtering with gold, the samples were viewed in the SEM. Close to the fracture line the sericin at nodes holding fibres together in a network (as described above) showed numerous very fine approximately parallel transverse cracks with a separation of about 1 μm. This appearance strongly indicates crazing. In addition, close to the fractured edge, crazing was also seen in the sericin coating of the relatively straight sections where the baves were not adhering to one another. Crazing regions in strained preparations were seen with the polarizing microscope as well as the SEM. Unstrained controls showed no evidence of crazing when examined with either microscope.

### Example 4

To demonstrate crazing in dried sericin a solution of sericin powder obtained commercially by degumming cocoons was prepared in deionised water (20% w/v). Drops of the prepared sericin solution were placed between two microscope slides and allowed to dry for a week at 20° C. After drying the slides were partially prized apart and the strained sericin remaining between the slides examined. Areas showed numerous fine parallel cracks clearly demonstrating crazing in dried sericin.

The existence of crazing in sericin shows that the dried material is in the glassy state. This is somewhat unusual for globular proteins air dried from aqueous solutions at ambient temperature, most of which dry to viscoelastic or resinous solids. This is because the glass transition temperature for dry globular proteins is normally well beneath ambient temperature.

To confirm that the sericin was in the glassy state at ambient temperature we used differential scanning calorimetry which gave a value of 88° C. for the glass transition temperature (T_{g}) of sericin flakes after drying in ambient air This is close to the value obtained by Ahn, J. S., H. K. Choi, et al. (2001). “Novel mucoadhesive polymer prepared by template polymerization of acrylic acid in the presence of silk sericin.” Journal of Applied Polymer Science 80(2): 274-280. The authors of this paper did not discuss the significance of this value for the functioning of sericin composites. An even higher value of 175° C. for the T_{g} was obtained after freeze drying of the preparation with a −50° C. trap followed by exhaustive drying over phosphorus pentoxide.

A search of the literature showed that the observed T_{g} of dried sericin was remarkably high compared with that for dry globular proteins but comparable to that of other dry fibrous proteins. The glass transition temperature of most globular proteins crystallized in their native state lies between −90 and −70° C. (see Vitkup, D., D. Ringe, et al. (2000). “Solvent mobility and the protein ‘glass’ transition.” Nature Structural Biology 7(1): 34-38), but increases to <30° C. on drying as a result of loss of plasticizing water (S Sitnitsky, A. E. (2002). “Modelling the “glass” transition in proteins.” Journal of Biomolecular Structure & Dynamics 19(4): 595-605 itnitsky 2002). The relatively small molecular weight (about 38 kDa) and highly hydrophilic character (see below) suggest that the sericin molecule may indeed be globular. Dry fibrous proteins appear to have considerably higher T_{g} for example 178° C. for silk fibroin threads (Agarwal, N., D. A. Hoagland, et al. (1997). “Effect of moisture absorption on the thermal properties of Bombyx mori silk fibroin films.” Journal of Applied Polymer Science 63(3): 401-410; 164° C. for zein and 209° C. for glutelin (see Di Gioia, L., B. Cuq, et al. (1999). “Thermal properties of corn gluten meal and its protein components.” International Journal of Biological Macromolecules 29: 341-350); and 220° C. for gelatin (see Sobral, P. J. A. and A. Habitante (2001). “Phase transitions of pigskin gelatin.” Food Hydrocolloids 15(4-6): 377-382).

However a high glass transition temperature is not the sole requisite for the production of a glass from a protein solution. It must also have a high solubility and loose water rapidly under ambient conditions to prevent the formation of crystals. Crystallisation certainly occurs when concentrated aqueous sericin solutions are allowed to evaporate slowly emphasizing the need for rapid drying to produce a glass. The ability to loose water readily under ambient conditions is also important to prevent plasticization. In addition for crazing to occur on straining, the sericin coat must be well bonded to the fibroin brins.

To investigate the properties of sericin further we first determined the solubility of sericin powder. Sericin showed a truly remarkable solubility, rapidly dissolving in water to give a strongly foaming solution that slowly gave a clear brown coloured solution containing in excess of 50 % w/v sericin. The foaming behaviour indicates strong surface activity. The 50% w/v sericin solution and considerably more dilute solutions were not colonized by bacteria, protozoa or fungi suggesting an antimicrobial action. This could result from their high surface activity of the sericin. Apart from the slow crystallization of highly concentrated solutions mentioned above, sericin solutions appeared to be remarkably stable, showing no tendency to gel even on prolonged storage.

We examined the published sequence of Bombyx mori sericin precursor (silk gum protein) SERI_BOMMO (P07856) for possible clues to its high solubility and rapid water loss. The remarkably high proportion of polar amino acid residues (87.65%) (serine 37.02%, threonine 8.2% and glycine 10.8%) helps to account for the high solubility. Further to this we used NetPhos v2.0 to examine the protein for putative eukaryotic phosphorylation sites as this post-translational modification could markedly enhance solubility and give rise to high surface activity. Remarkably 78.5% of the serine residues and 47% of the tyrosine residues were potentially phosphorylated but only 6.2% of the threonines. The possible phosphorylation sites are fairly uniformly distributed throughout the length of the protein except for a marked absence in the highly hydrophobic putative signal peptide and a small region showing a marked concentration fairly close to the middle of the sequence. It is also to be noted that phosphorylation in addition to increasing solubility and surface activity would greatly increase the weight of serine and tyrosine side chains and this would be likely to reduce the mobility and hence increase the glass transition temperature compared with unphosphorylated proteins. As in DNA (reported in Egli, M., V. Tereshko, et al. (1998). “X-ray crystallographic analysis of the hydration of A- and B-form DNA at atomic resolution.” Biopolymers 48(4): 234-252), the phosphorylation of sericin may also enhance the thermodynamics of water loss. The predicted isolectric point of 7.7 suggests that the protein molecule carries little net charge at physiological pH values. This could be advantageous as it would allow them to approach one another closely facilitating the formation of hydrophobic and van der Waals interactions between protein molecules. This in turn would promote condensation of the molecules into a glass A more or less globular tertiary structure and markedly hydrophilic surface of sericin is also likely to facilitate rapid water loss compared with fibrous proteins as the existence of hydrophobic blocks exposed to the surface in the latter provides for the attachment of ice-like shells of tightly bound water (clathrate).

Energy dispersive X-ray analysis was used to discover whether the glass transition temperature of sericin is enhanced naturally by binding heavy elements. A 5% w/v solution of sericin in water was exhaustively dialysed against deionized water. The thin sericin films were cast on exhaustively washed polystyrene weighing boats. Fragments of the resulting brittle film were mounted on formvar-carbon films on copper grids and subjected to energy dispersive X-ray analysis using an H-7000 Hitachi microscope fitted with an EDAX analyzer. An unfocused beam was used during X-ray collection. Only two elements could be detected from the sericin, a fairly prominent peak corresponding to the calcium K line and a much smaller peak corresponding to the K line of silicon. Control spectra were collected from the formvar-carbon films and showed no detectable elements. These observations suggest that calcium and silicate is tightly bound to the sericin though the binding of these elements to another component in the sericin powder as supplied cannot be ruled out. However sericin is extremely rich in serine and this amino acid is known to bind silicate in other proteins. Although precise quantification was not possible the EDX spectra show considerably less silicon than calcium suggesting that the major part of the calcium is not present as calcium silicate but is probably directly bound to the protein. Thus it seems likely that silicate and calcium are directly bound to the protein and that these elements play a part in increasing the glass transition temperature of the sericin in vivo.

In an attempt to define the structure of sericin flirter we first used the repeat detecting program Radar to confirm the existence of a 40 residue contiguously repeated consensus sequence (GSSTSGG..SSTYGYSSNSRDGSVSSTGSSSNIDSNSNSX where X is A or V) in the complete published sericin precursor sequence SERI_BOMMO (P07856). The sequence showed a repetitive block copolymeric structure consisting of 6 rather well conserved repeats and one partial repeat of the consensus sequence (see FIG. 5).

We then used a SIB BLAST search to look on the PDB data base for proteins showing significant similarity to the consensus repeat. The best hit was 1INA ICEN_PSESY Ice nucleation protein (inaz) with a score 89 and a highly significant e value (5e⁻¹⁸). We then used LALIGN to determine multiple overlaps between these two proteins (see Fig.). Significant similarity is seen in both polyserine and relatively serine-poor regions. These results demonstrate a remarkable similarity between ice nucleation protein and sericin. This similarity may have functional significance as the absence of an ice-like water shell may be important for the function of both proteins.

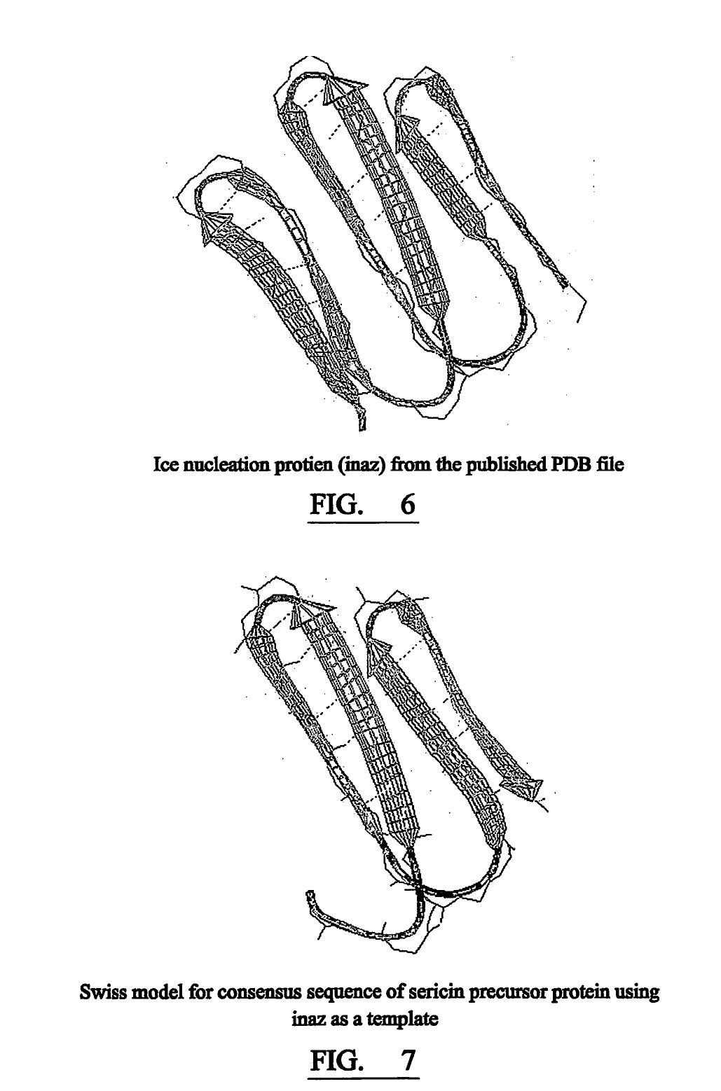

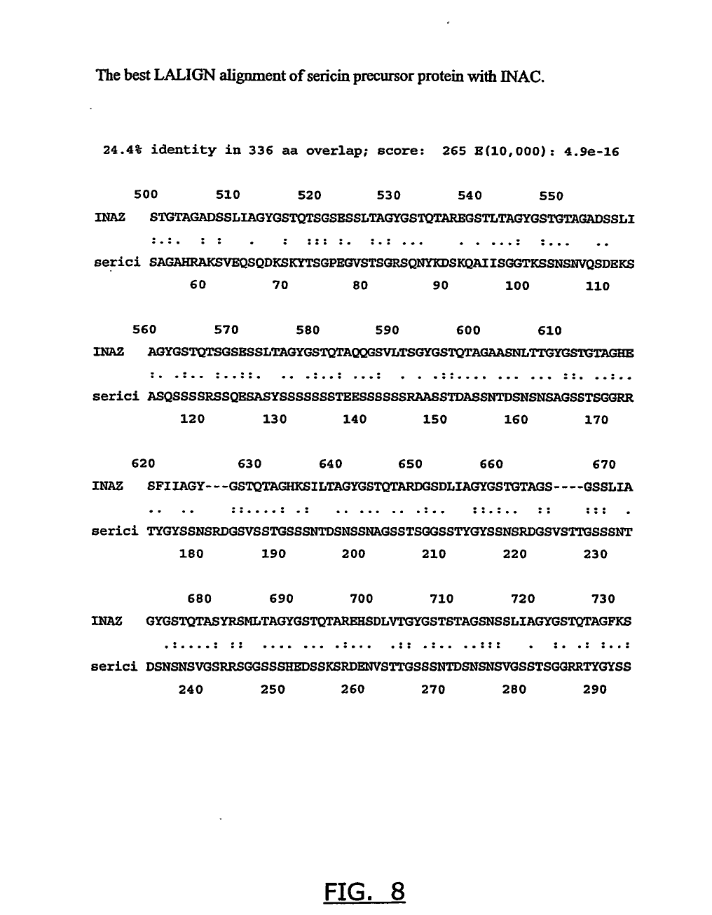

We then used SWISS-MODEL to model the 40 mer consensus repeat sequence of sericin using the published PDB coordinates of ice nucleation protein (see FIG. 6) as a template. The resulting energy minimized model for the consensus sequence showed four repetitions of alternating turn and strand with antiparallel beta sheets formed between the first and second and third and fourth strands. The slightly twisted beta sheets lay approximately parallel to one another (see FIG. 7). The longer sequence for inaz showed an additional anti-parallel beta sheet lying approximately parallel to the other beta sheets. These observations suggest that the repetitive region of the sericin precursor protein is constructed solely from an alternation of approximately 24 turns with 24 beta strands showing a repeated up/down/up/down arrangement. Adjacent beta strands hydrogen bond to give rise to 12 anti-parallel beta sheets. Although the tertiary structure cannot be specified in detail at present, the alternate turn/strand structure is clearly more compact than that of the more extended chains of an alpha helical or collagenous fibrous protein. A beta spiral or beta propeller structure is possible.

The example demonstrates that silkworm silk cocoons are composite materials toughened and stiffened by dried sericin present in the glassy state. Short regions of poorly orientated material functions as nodes sticking together a framework produced by extremely tough tension members formed from brins containing well-orientated fibroin surrounded by a thin sericin coat. The short, poorly orientated regions make the bave weak when it is unwound from the cocoon but are designed to provide considerable toughness to the composite network by crazing preferentially when the network is strained. The high solubility, rapid water loss and relatively high glass transition temperature for a non-fibrous protein probably assist in the formation of a crazing glass. These properties in turn appear to be dictated by the very high proportion of polar amino acids, a probable extensive phosphorylation of the protein, its lack of net charge at physiological pH and its relatively compact structure. We have also demonstrated that the toughness of a fibroin meshwork can be considerably increased by substantially filling the interstices between the fibres with sericin in the glassy state.

1-78. (canceled)

79. An article comprising a plurality of elongate elements embedded in a bulk matrix, said bulk matrix comprising a first glassy protein.

80. The article according to claim 79 wherein said first glassy protein comprises at least 5 mol % serine.

81. The article according to claim 79, wherein said first glassy protein comprises a protein selected from the group of repetitive block proteins consisting of (a) sericin; (b) Pseudomonas seringae ice nucleation protein INAZ; (c) Drosophila putative chitin binding protein QVEL9 (d) homologs of (a), (b) and (c); and (e) analogs of (a), (b) and (c).

82. The article according to claim 79, wherein said bulk matrix further comprises at least one second glassy protein.

83. The article according to claim 82, wherein said second glassy protein has a same amino acid sequence as said first glassy protein but differs from said first glassy protein by at least 25% in its degree of phosphorylation and/or glycosylation.

84. The article according to claim 79, wherein said bulk matrix further comprises a polymer material.

85. The article according to claim 79, further comprising a coating on a surface of the article that is substantially impermeable to water and water vapor.

86. The article according to claim 79, wherein said elongate elements are selected from the group of fibers consisting of (a) glass fibers, (b) carbon fibers, (d) carbon nanotubes, (e) montmorilonite clay particles, (f) polymers and (g) fibrous proteins.

87. The article according to claim 86, wherein said fibrous proteins are selected from the group of proteins consisting of (a) spider silk proteins; (b) analogs of spider silk proteins; (c) silk-worm proteins; (d) analogs of silk-worm proteins; (E) regenerated silk protein; (f) mixtures of two or more of (a) to (e), and (g) fibrous proteins.

88. A method of manufacturing an article, comprising the steps of:
(a) preparing, in an aqueous solvent and at a concentration of at least 20 wt %, a solution of a protein capable of forming a glassy state;
(b) contacting a plurality of elongate elements with said solution so that said elongate elements are substantially wetted by said solution;
(c) substantially filling interstices between said elongate elements with said solution; and
(d) drying the product of step (c);
wherein said article comprises elongate elements embedded in a bulk matrix formed by said solution on drying and comprising a glassy protein.

89. The method according to claim 88 wherein step (a) comprises preparing said solution by dissolving substantially purified sericin in an aqueous solvent to a concentration of at least 20 wt %.

90. The method according to claim 88, wherein step (c) is performed by exerting pressure on said solution in the presence of said elongate elements.

91. The method according to claim 88 comprising an additional step prior to step (d), wherein said additional step comprises shaping said plurality of elongate elements over the surface of a mold.

92. The method according to claim 88 comprising an additional step (e) that follows step (d), wherein said additional step comprises applying a coating that is substantially impermeable to water and water vapor.

93. The method according to claim 88, further comprising a step of treating said solution of the protein capable of forming a glassy state, with a solution containing silicate ions.

94. The method according to claim 88, further comprising a step of treating said solution of the protein capable of forming a glassy state, with a solution containing calcium ions.

95. A method of manufacturing a laminate comprising the steps of:
(a) preparing, in an aqueous solvent and at a concentration of at least 20 wt %, a solution of a protein capable of forming a glassy state;
(b) preparing a plurality of layers of substantially planar sheets, said substantially planar sheets comprising a plurality of elongate elements;
(c) contacting said substantially planar sheets with said solution so that said elongate elements are substantially wetted by said solution;
(d) substantially filling interstices between said elongate elements and interstices between the sheets of said plurality of substantially planar sheets, with said solution; and
(e) drying said substantially planar sheets.
